(19) United States
(12) Patent Application Publication
(10) Pub. No.: US 2009/0216195 A1
(43) Pub. Date: Aug. 27, 2009

(54) Surgical Needle or Introducer with Precise Penetrator

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/394,168

(22) Filed: Feb. 27, 2009

Related U.S. Application Data
(60) Provisional application No. 61/067,326, filed on Feb. 27, 2008.

(51) Int. Cl. A61B 17/34

(52) U.S. Cl. 604/157; 604/161

(57) ABSTRACT

A penetrating instrument and method for piercing a desired tough or elastic tissue to a controlled and predetermined depth so as to avert any danger of damaging organs or tissues more deeply disposed. The instrument may comprise an introducer needle capable of independently penetrating bodily tissue and having a central hollow channel wherein a piercing needle is axially movable therein. The piercing needle may comprise a distal tip that is preferably fully disposed within the central hollow channel when the piercing needle is in a retracted position, but the piercing needle is capable of being deployed a predetermined distance beyond the distal end of the central hollow channel when disposed in an extended/piercing position. The introducer needle may be advanced through tissue until selected, problematic, or tough tissue is encountered. Subsequently, the piercing needle may be deployed and retracted, as necessary, to facilitate advancement of the instrument through the specific tissue.

### CROSS REFERENCE TO RELATED APPLICATIONS

This application claims the benefit of provisional patent application Ser. No. 61/067,326, filed with the USPTO on Feb. 27, 2008, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to needles, more specifically, the present invention relates to surgical needles used in combination for making precise penetrations of a predetermined distance into resistive or tough tissue.

2. Background Art

Penetrating instruments are widely used in medical procedures to gain access to anatomical cavities ranging in size from the abdomen to small blood vessels, such as veins and arteries, epidural, pleural and subarachnoid spaces, heart ventricle and spinal and synovial cavities, with access being established via an outer sleeve or cannula positioned during penetration into the cavity with the penetrating instrument. Such penetrating instruments include a penetrating member having a sharp tip or point to pierce or penetrate the tissue forming the cavity wall, and the force required to penetrate the cavity wall is dependent upon the type and thickness of the tissue of the wall. Once the wall is penetrated, it is desirable to prevent the sharp tip of the penetrating member from inadvertent contact with or injury to tissue or organ structures in or forming the cavity, and a particular problem exists where substantial force is required to penetrate the cavity wall or the cavity is very small in that, once penetration is achieved, the lack of tissue resistance may result in the sharp tip traveling too far into the cavity and injuring adjacent tissue or organ structures.

A problem with many penetrating instrument systems is that the ends of the system components are not appropriately shaped to puncture the skin and the intervening tissue between the skin and the treatment site. Some systems have blunt, flat ends. Some systems have some tapered components which would ease their penetration of tissue to the treatment site, but their designs make them difficult to use. Furthermore, many penetrating instruments are tasked with the job of penetrating multiple tissue planes of varying thickness and resistance. Hence, the penetrating instrument may be well designed to penetrate one tissue safely, but the design is not safe to penetrate the next tissue plane. An example of this would be a needle with a dulled tip geometry that can penetrate the skin without risk of injuring underlying organs but this same dull tip geometry prevents penetration of a more pliable membrane such as the peritoneum.

One example of a prior art trocar is the device disclosed in U.S. Pat. No. 5,824,002. This patent discloses a “transition-less” trocar, that is, a trocar which provides a smooth geometric transition between the tip of the obturator to the end of the cannula so that the trocar can be inserted with reduced force compared to trocars having an abrupt geometric transition while reducing the potential for damaging tissues. However, there is still a need in the art to further reduce the potential for damaging tissue, sutures, and the instruments themselves as such devices that more easily penetrate tissue without substantial force are typically sharp and more prone to injuring organs.

One of the limitations of many prior art safety penetrating instruments is that the safety probes may produce an irregular surface or profile with the sharp tips of the penetrating members during penetration of tissue resulting in increased resistance during penetration of a cavity wall, trauma and damage to tissue, and possible jamming and trapping of tissue. Another limitation of many prior art safety penetrating instruments is that very small or narrow anatomical cavities cannot be penetrated without protrusion of the safety probes or shields from the sharp tips of the penetrating members. A further limitation of many prior art penetrating instruments is that the penetrating members cannot be automatically retracted, with or without the safety probes or shields within the outer sleeves upon penetration into an anatomical cavity. Many prior art penetrating instruments are also limited in that the penetrating members cannot be automatically retracted to safe, protected positions wherein the sharp tips of the penetrating members are disposed within the instruments upon penetration into anatomical cavities and thereafter selectively moved to various extended positions with the sharp tips protruding into the cavities for performing various surgical procedures.

While generally successful, the sharpened distal tip of a penetrating instrument presents a risk of injury to underlying body structures when the tip of such an instrument is introduced through tissue. In order to avoid such injury, it is important that the sharpened distal tip of the penetrating instrument be stopped or somehow protected immediately after the tip penetrates through the chosen tissue.

One such approach for protecting tissue from the penetrating tip relies on a spring-loaded shield mounted concentrically over the piercing member. The shield is drawn proximally by contact with tissue as the piercing member and outer cannula are advanced through the abdominal wall. As soon as the piercing member enters a body cavity, however, the shield springs back over the sharpened tip of the piercing member to protect the underlying body organs and surrounding tissue from damage. Such an approach has been generally successful, but suffers from certain drawbacks. In particular, in some cases the shield may become jammed or may simply not respond quickly enough to cover the obturator before contact with body structures in the underlying tissue occurs.

Even with such a prior art device responding as intended, the sharpened piercing member tip is still able to penetrate beyond the tissue by a short distance, approximately 1 cm to 3 cm, before the shield redeploys over the entire length of the piercing member. Thus, there still remains a risk of injury during this length of unprotected travel. An alternative approach for protecting the penetrating instrument tip relies on a spring-loaded core within the piercing member, where the core retracts as the piercing member is advanced. Although an improvement over the external shield, the core can still become jammed.

For these reasons, it would be desirable to provide alternative apparatus and methods for protecting a patient during introduction of a penetrating system during surgical procedures, such as laparoscopic procedures and vaginal surgery. It would be further desirable to provide protection which is responsive to the inherent demand for improved safety and control as a sharpened distal tip of a penetrating instrument enters a selected body tissue. Such apparatus and methods should be reliable, easy to implement, and relatively inexpensive to produce.

The present invention is generally characterized as a penetrating instrument generally comprising an introducer needle and a piercing needle slidably disposed within the introducer needle and an optional introducer member that may be mounted on the proximal end of the introducer needle. The piercing needle is movable to an operative state with the sharp distal tip of the piercing needle protruding beyond a distal end of the introducer needle to assist in penetrating tissue, wherein the piercing needle may be retracted within the introducer needle upon penetration through the specified tissue. The operator may chose to penetrate with the introducer needle or the piercing needle, based on the characteristics of the resisting tissue.

These and other objects and advantages of the present invention will become apparent from the following description of the preferred embodiments taken in conjunction with the accompanying drawings wherein identical reference numbers indicate identical parts or parts providing identical functions.

### BRIEF SUMMARY OF THE INVENTION

In accordance with one embodiment, a penetrating instrument comprising an introducer member, an introducer needle having a proximal end and a distal end, the introducer needle in communication with and distally extending from the distal surface of the introducer member, a piercing needle having a distal tip, the piercing needle in slidable communication with the introducer needle, and an actuating member connected to the piercing needle, the actuating member axially advances the piercing needle a predetermined distance relative to the introducer needle wherein the piercing needle is actuated between a retracted position and a deployed position.

The scope of the present invention also includes a method for penetrating selected tissue, comprising the steps of providing a penetrating instrument comprising an introducer member, an introducer needle having a proximal end and a tapered distal end, the introducer needle in communication with and distally extending from the distal surface of the introducer member, a piercing needle having a distal tip, the piercing needle in slidable communication with the introducer needle, and an actuating member connected to the piercing needle, the actuating member axially advances the piercing needle a predetermined distance relative to the introducer needle wherein the piercing needle is actuated between a retracted position and a deployed position, the retracted position comprising the distal tip of the piercing needle disposed proximal to the distal end of the introducer needle, the deployed position comprising the distal tip of the piercing needle disposed the predetermined distance distal to the distal end of the introducer needle, wherein the tapered distal end of the introducer needle allows for independent penetration of tissue by the introducer needle when the piercing needle is disposed in the retracted position, wherein the actuating member is selected from the group consisting of a manual grip, a lever, a spring, and or a motor activator and the actuating member further provides for retraction of the piercing needle from the deployed position to the retracted position, applying an advancement force to the introducer member, advancing the introducer needle through secondary tissue until the distal end of the introducer needle is disposed immediately adjacent to the selected tissue, wherein the piercing needle is disposed in the retracted position, applying an activation force to the piercing needle, and advancing the piercing needle from the retracted position to the deployed position, wherein the piercing needle is advanced the predetermined distance into the selected tissue.

### BRIEF DESCRIPTION OF THE DRAWINGS

FIG. 1 depicts a side view of an embodiment of the present invention disposed immediately adjacent a selected tough tissue to be penetrated.

FIG. 2 depicts a side view of the embodiment of FIG. 1 with the piercing needle in a deployed position acting to penetrate the selected tough tissue.

FIG. 3 depicts a side view of the embodiment of FIG. 2 wherein the entire penetrating instrument advances along the penetration path initiated by deployment of the piercing needle.

FIG. 4 depicts a partial side view of another embodiment of the present invention.

FIG. 5 depicts a partial perspective view of the embodiment of FIG. 4 of the present invention.

FIG. 6 depicts a close up, partial perspective view of the embodiment of FIG. 4 of the present invention.

### DETAILED DESCRIPTION OF THE INVENTION

Surgeons use numerous sizes and shapes of needles or introducers to penetrate bodily tissue. One of the most difficult tasks a surgeon may face is the controlled penetration of tough, resistant tissues. As substantial force is necessary to penetrate tough tissue, this force often results in continued movement of the needle or introducer following the initial penetration. This continued movement often results in injury to underlying organs, tissues, or the user. Currently available solutions only utilize needle geometry and/or video technology in an attempt to alleviate this problem.

Due to the delicate nature with which penetrating instruments are used, it is highly desirable to minimize the damage to surrounding tissue and organs during the insertion and removal process of such devices. Thus, one goal of the present invention is to provide a penetrating instrument and a method of using a penetrating instrument that minimizes or eliminates the potential for damaging surrounding tissues or organs. Another desirable feature of a penetrating instrument is that it requires as little force or effort by the surgeon as possible while in use. Thus, another goal of the present invention is to provide a penetrating instrument and a method of using a penetrating instrument that requires less force and effort by the surgeon. In addition, another aspect of the present invention provides a penetrating instrument that facilitates the piercing and advancement through a tough selected tissue or elastic tissue when substantial resistance or elasticity is met, the piercing and advancement of the present inventive instrument is more controlled and less likely to damage surrounding tissue as compared to the devices and methods of the prior art.

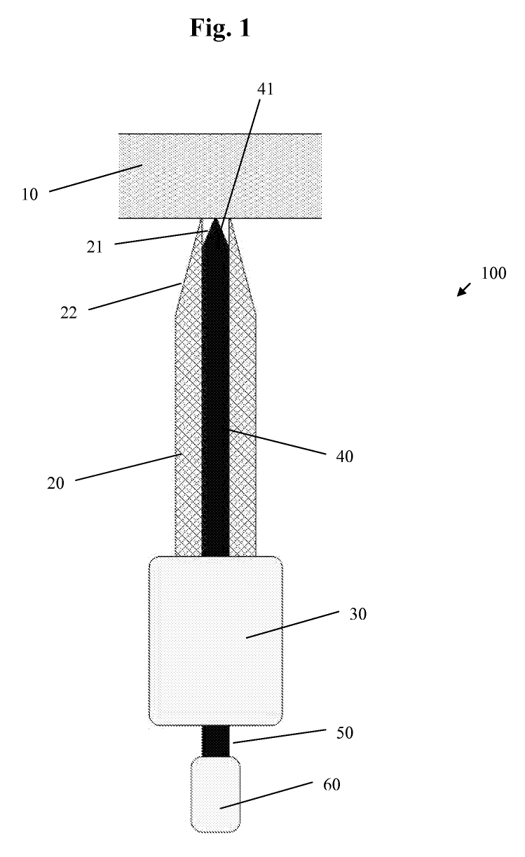

FIGS. 1-3 depict highly schematic side views of one embodiment of a penetrating instrument 100 of the present invention. This embodiment may include an introducer needle 20 and an introducer member 30. The introducer needle 20 may be in communication with the introducer member 30, wherein the introducer needle 20 is independently capable of penetrating bodily tissue. In the embodiment depicted in FIGS. 1-3, the introducer needle 20 is shown disposed on the distal surface of the introducer member 30. In other possible embodiments, the introducer needle 20 may enter or entirely pass through the introducer member 30 wherein the introducer member 30 may be used to facilitate the manual advancement of the introducer needle 20 through the bodily tissue. The introducer member 30 is typically adapted to be held by the surgeon, for example, during insertion or withdrawal of the introducer needle 20. In this manner, the introducer member 30 may typically provide a surface upon which the surgeon may grip and push when inserting the introducer needle 20 through tissue being penetrated and also provides a means for grasping the introducer needle 20 when removal of the penetrating instrument 100 is desired. The introducer needle 20 may additionally comprise a central hollow channel 21 and a tapered distal tip 22. The incorporation of the tapered distal tip 22 permits relatively easy insertion of the introducer needle 20 through, for example, the normal muscle and fascia of a patient with minimal force and minimal damage to the tissue penetrated and minimal damage to the surrounding internal tissues and organs.

In one embodiment, the central hollow channel 21 of the introducer needle 20 may contain or otherwise be in communication with a piercing needle 40 disposed in slidable communication with the introducer needle 20. The piercing needle 40 or an extension 50 of the piercing needle 40 may extend into or through the introducer member 30. As depicted in FIGS. 1-3, the proximal end of the piercing needle 40 or extension 50 of the piercing needle 40 may further comprise an actuating member 60 that may assist in deployment and/or retraction of the piercing needle 40 from within the introducer needle 20.

FIGS. 1-3 illustrate a retractable safety-penetrating instrument 100 according to one embodiment of the present invention that includes an introducer needle 20, an introducer member 30 mounted to the introducer needle 20, and a piercing needle 40 disposed within or otherwise about the introducer needle 20. Introducer needle 20 is preferably made of a medical grade material, such as stainless steel, and has an outer diameter or size dependent upon the surgical procedure to be performed and the anatomical tissue to be penetrated. The introducer needle 20 may have a distal end terminating at a tapered distal tip 22 for penetrating anatomical tissue. The introducer needle 20 may be either solid or possess a central hollow channel 21, and its tapered distal tip 22 may have various hollow or solid geometrical configurations. As shown in the embodiment of FIG. 1, the introducer needle 20 may be hollow and have a tapered distal tip 22 disposed at an angle with a longitudinal axis of the introducer needle 20. The introducer needle 20 may comprise an elongated body that may be cylindrical or have any desirable configuration in cross-section. The introducer needle 20 may extend proximally to communicate with the introducer member 30 and the introducer needle 20 may pass through an aperture in a distal surface of the introducer member 30.

The piercing needle 40 may comprise a solid material shaft having a tapered distal tip 41 that provides for initial precise penetration of a selected or tough tissue 10. The distal tip 41 of the piercing needle 40 may initially be disposed in a retracted position (see FIG. 1) inside or flush with the distal tapered tip 22 of the introducer needle 20. With the introducer needle 20 being independently capable of tissue penetration without the assistance of the piercing needle 40 being disposed therein, the piercing needle 40 is preferably disposed entirely within or flush with the tapered distal tip 22 of the introducer needle 20 until substantial resistance is met by the introducer needle 20. The piercing needle 40 may then be used to provide further assistance to the surgeon to safely advance the penetrating instrument 100 through tough or selected tissue 10. A variety of needle configurations, for both the introducer needle 20 and piercing needle 40, may be employed and have several degrees of tip sharpness. Each tip may be optimized for certain locations of anatomy as well as different levels of puncture force depending on the surgical technique being used. Any tip geometries and needle materials known within the art may be incorporated into both the introducer needle 20 and piercing needle 40 of the present penetrating instrument 100.

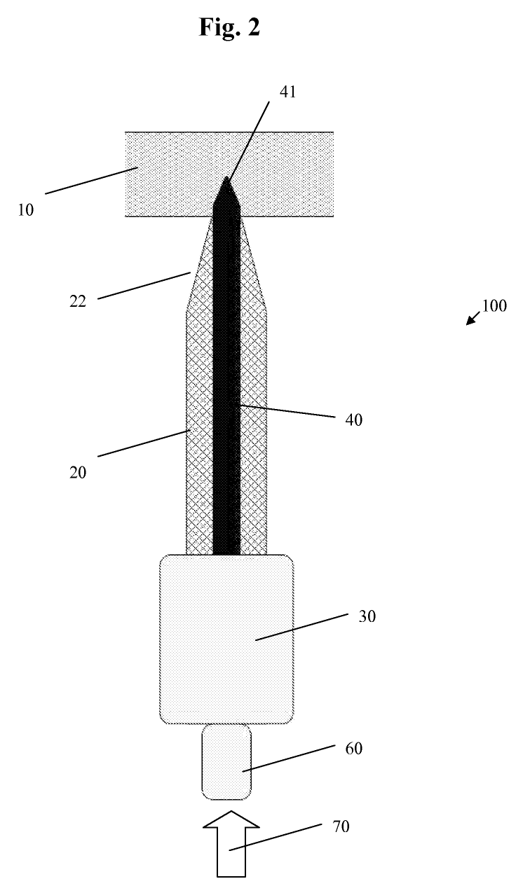

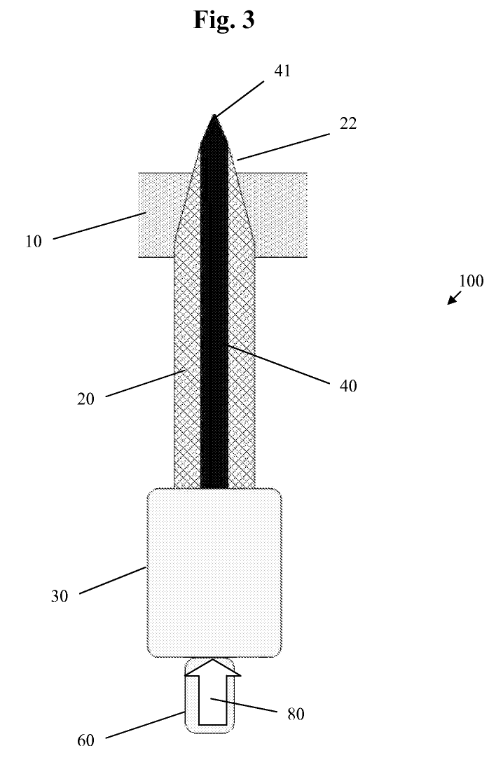

Both the introducer needle 20 and the piercing needle 40 are each independently capable of piercing or penetrating the majority of bodily tissues and are distinct from needles disposed within trocars, obturators, sheaths or catheters of the prior art. When using the above needles in combination, both the safety and effectiveness of penetrating tough and or elastic bodily tissue 10 is greatly increased. A surgeon may advance the introducer needle 20 until meeting substantial resistance from a specific tough tissue or elasticity from an elastic tissue 10. To minimize the potential damage to surrounding tissue by too forceful of an advancement of the introducer needle beyond the tough or elastic tissue 20, the piercing needle 40 may be motivated from a retracted position to a deployed position by an activation force 70, as depicted in FIGS. 1-2. The distance of maximum deployment of the piercing needle 40 may be set to a distance just beyond the thickness of the tough or elastic tissue 10 such that the advancement of the piercing needle 40 is capable of penetrating the tough or elastic tissue 10 without any advancement of the introducer needle 20 and without risk of continued travel to underlying tissues or organs. Following this penetration, the piercing needle 40 may be retracted and the introducer needle advanced through the tough or elastic tissue 10 with minimal force and maximal safety. The piercing needle 40 may then be retracted and re-deployed as often as necessary. Additionally or alternatively after deployment of the piercing needle 40, an advancement force 80 may be applied to the penetrating device 100 to further advance the device 100 through the tough tissue 10 (see FIG. 3).

The present inventive penetrating instrument 100 may further comprise an actuating member 60 (generally depicted in FIGS. 1-3) for advancing the piercing needle 40 from a retracted position (see FIG. 1) to a distally extended or deployed position (see FIG. 2). Such an actuating member 60 may include but is not limited to all one or more manual grip surfaces, levers, springs, motor activators, and like activation means known in the art for advancing a needle through tissue. The actuating member 60 advances the piercing needle 40 from its retracted position (see FIG. 1) into its deployed position (see FIG. 2) with sufficient activation force 70 to pierce the tough or selected tissue 10. The application of an activation force 70 on the actuating member 60 and/or piercing needle 40 may be accomplished by but is not limited to manual advancement of the one or more grip surfaces, the pressing of a button, squeezing of a trigger, or other means that may activate a lever, release a spring, or other coiled tension device, and the like. In one embodiment, the actuating member 60 may comprise a lever mechanism which when squeezed acts to advance the piercing needle 40 into its deployed position, as shown in FIG. 2. Likewise, another embodiment of the actuating member 60 may comprise a spring loaded piecing needle 40 that, when activated, advances the piercing needle 40 into the adjacent tissue 10 with a preloaded amount of force 70. All embodiments of the actuating member 60 may also participate in the retraction or “re-loading” of the piercing needle 40 into its retracted position after each respective deployment thereby allowing for one or more additional penetration cycles. The present inventive penetrating instrument 100 and method may utilize as many penetration cycles as are necessary to achieve penetration of the tough or selected tissue 10 encountered during the selected surgical procedure.

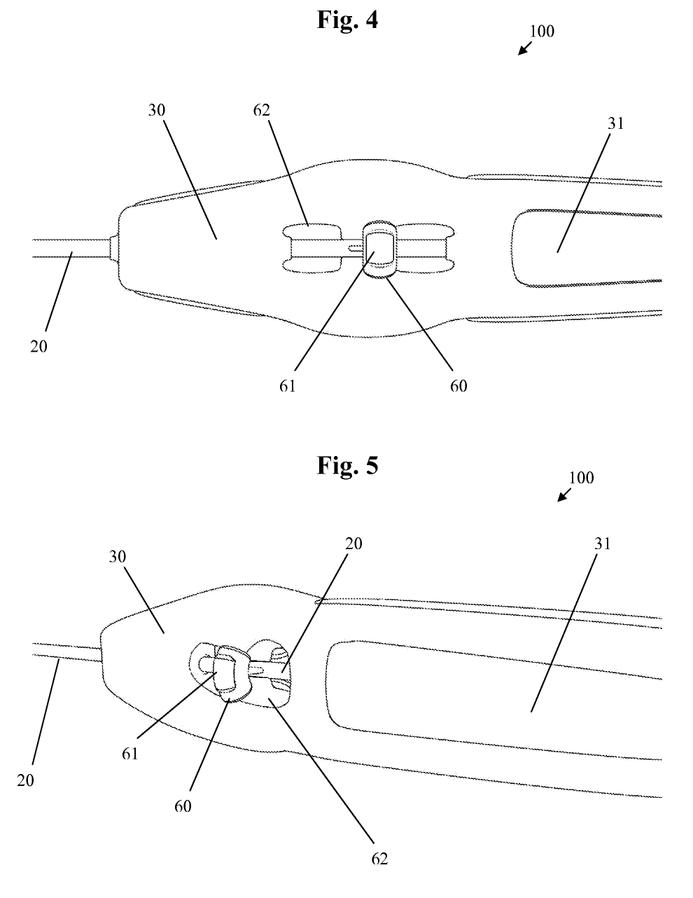

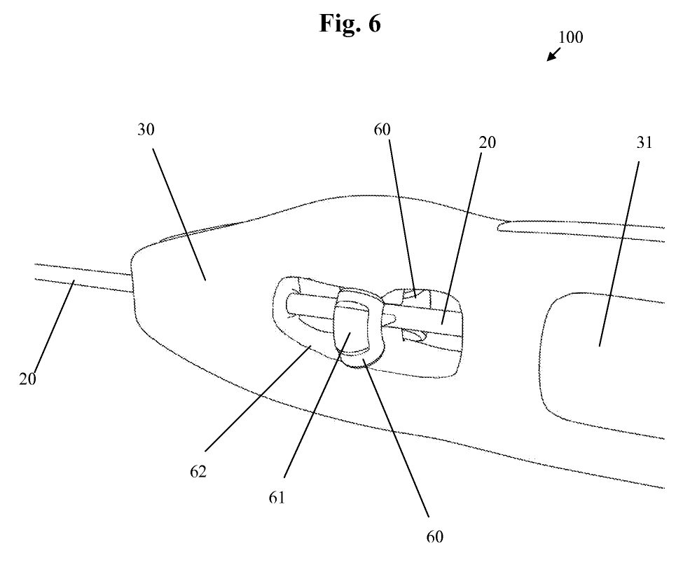

FIGS. 1-3 generally depict the introducer member 30 of the present invention. In varying embodiments of the present invention, other components of the penetrating instrument 100 may be disposed upon, within, or through the introducer member 30. In a preferred embodiment as depicted in FIGS. 4-6, the structural components of the selected actuating member 60 may be contained within the introducer member 30 and be accessible to a surgeon while the surgeon is grasping the introducer member 30. In such an embodiment, the introducer member 30 may provide a convenient location for grasping the penetrating instrument 100 while the surgeon manipulates the actuating member 60 and performs the surgical procedure. The introducer member 30 may further comprise one or more grip portions 31 to facilitate the grasping of the introducer member 30. The grip portions 31 may comprise different materials, surfaces, or textures to aid in surgical manipulation of the penetrating device 100 during a surgical procedure.

As further depicted in FIGS. 4-6, the introducer member 30 may comprise a central aperture 62 wherein the actuating member 60 may be disposed. The central aperture may provide access to the introducer needle 20 and/or the piercing needle 40 that are exposed therein. The actuating member 60 may be in communication with the piercing needle 40 within the central aperture 62. Distal manipulation of the actuating member 60 via an activation force 70 may move the piercing needle 40 from a retracted position to a deployed position relative to the introducer needle 20. To assist in the application of the activation force 70 and to provide tactile feedback, the actuating member 60 may have at least one contact surface 61 disposed on the exterior of the actuating member 60. In a preferred embodiment, the actuating member 60 may be pushed inward toward the introducer needle 20 and/or piercing needle 40 to unlock the position of the actuating member 60 and allow for slidable axial movement thereafter. With the actuating member 60 depressed and unlocked, the actuating member 60 may then be distally translated or slid via an activation force 70 along the axis of the introducer needle 20 and/or piercing needle 40. After precise penetration, the actuating member 60 may be translated or slid proximally along the axis of the introducer needle 20 and/or piercing needle 40. Such proximal movement of the actuating member 60 may motivate the piecing needle 40 from a deployed position (FIG. 2) to a retracted position (see FIG. 1). The proximal movement of the actuating member 60 may result from a user's manual application of force, a biased spring return force, or any other motivating force generation means known within the art.

In use and as depicted in FIG. 1, a surgeon or other practitioner may advance the penetrating instrument 100 through secondary bodily tissue until substantial resistance is met, such as when encountering either selected or tough tissue 10. In such a position, the tapered distal tip 22 of the introducer needle 20 may immediately abut the specific tissue 10. The distal tip 41 of the piercing needle 40 is preferably in a retracted position and either fully disposed within the introducer needle 20 or flush with the tapered distal tip 22 of the introducer needle 40. The piercing needle 40 may preferably be disposed within a central hollow channel 21 of the introducer needle 20 as depicted in FIGS. 1-3.

As depicted in FIG. 2, application of an activation force 70 on the actuating member 60 may motivate the piercing needle 40 from a retracted position to a piercing or deployed position. The deployment of the piercing needle 40 occurs over only a fixed, pre-determined travel distance from beyond the tapered distal tip 22 of the introducer needle 20 so as to facilitating safe and precise penetration. Such a limitation imposed on the piercing needle's 40 travel distance provides for a known and highly accurate depth of tissue 10 penetration and averts the dangers of piercing, tearing, or otherwise damaging surrounding organs and other tissues more deeply disposed. The deployment of the piercing needle 40 via the activation force 70 may comprise a variety of modalities including but not limited to manual deployment or deployment facilitated by a spring, lever, motor activator, and the like. Such activation of the piercing needle 40 may be repeated as necessary with a second activation force, third activation force, and the like. A retraction force may proximally retract the piercing needle 40 after each respective activation force 70 is applied. The retraction force may be provided in the form of a user's manual force, a spring or other coiled force, a lever action, motor activator, and the like.

The activation force 70 necessary to penetrate the specific tissue 10 may be altered by the user's choice of tip geometry including but not limited to the tip style, size, and width of the needle. In this manner, the tip geometry may be optimized to decrease the necessary penetration force without increasing the risk of injury. Additionally, the actuating member 60 may also function to withdraw or retract the piercing needle 40 to allow for repeated activations of the piercing needle 40 as necessary and/or to protect surrounding tissue once a successful and precise penetration has occurred.

With the distal tip 41 of the piercing needle 40 in a fully deployed position into the tissue 10 (see FIG. 2), the entire penetrating instrument 10 may then more easily be advanced into the tissue 10 (see FIG. 3) via application of an advancement force 70 on the penetrating instrument 100. In this manner, the piercing needle 40 serves to initiate the penetration and creation of a pathway through the selected or tough tissue 10 thereby facilitating later advancement of the entire penetrating instrument 100 into the precise penetration hole generated by the piercing needle 40 upon its activation. The advancement force 70 may be applied to the penetrating instrument 10 and its introducer needle 20 only after the piercing needle 40 has been activated at least one time into its deployed position and initiated penetration through the selected tissue 10. Each activation of the piercing needle 40 is limited to a pre-determined distal travel distance to provide for a controlled, safe advancement of the instrument 100 into the desired tissue 10. The process described herein may be repeated as necessary to achieve a safe and precise penetration path of a pre-determined distance into a desired tissue 10.

In alternate embodiments, the introducer needle 20 may not comprise a central hollow channel 21 and the piercing needle 40 may not be disposed therein. In this manner, the scope of the present invention is not necessarily limited to a piercing needle 40 disposed within an introducer needle 20. The piercing needle 40 may be in communication with and axially slide along the introducer needle 20 wherein the piercing needle 40 may sit on top of the introducer needle 20 (e.g. saddle configuration) or reside within a feature, such as a groove, disposed on the outer surface of the introducer needle 20. In these examples of alternate embodiments, the introducer needle 20 may transport the piercing needle 40 to a location immediately adjacent a selected or tough tissue 10 via a number of structural means. Once the penetrating device 100 is so positioned, the functionality of the penetrating device 100 and the method steps associated therewith are essentially the same as disclosed above. The piercing needle 40 may be activated and deployed a pre-determined distance into the tissue 10 to provide a safe and efficient means of advancing the penetrating instrument 100 into the selected tissue 10.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the examples given.

What is claimed is:

1. A penetrating instrument, comprising:
an introducer needle having a proximal end and a distal end;
a piercing needle having a distal tip, said piercing needle in slidable communication with said introducer needle; and
an actuating member connected to said piercing needle, said actuating member axially advances said piercing needle a predetermined distance relative to said introducer needle wherein said piercing needle is actuated between a retracted position and a deployed position.

2. The penetrating instrument of claim 1, wherein said retracted position comprises said distal tip of said piercing needle disposed proximal to said distal end of said introducer needle.

3. The penetrating instrument of claim 1, wherein said deployed position comprises said distal tip of said piercing needle disposed said predetermined distance distal to said distal end of said introducer needle.

4. The penetrating instrument of claim 1, wherein said distal end of said introducer needle is tapered allowing for independent penetration of tissue by said introducer needle when said piercing needle is disposed in said retracted position.

5. The penetrating instrument of claim 1, wherein said actuating member is selected from the group consisting of a manual grip, a lever, a spring, and a motor activator.

6. The penetrating instrument of claim 1, wherein said actuating member provides for retraction of said piercing needle from said deployed position to said retracted position.

7. The penetrating instrument of claim 1, wherein said introducer needle comprises a central hollow channel in which said piercing needle is disposed and axially movable therein between said retracted position and said deployed position.

8. The penetrating instrument of claim 1, wherein said piercing needle is disposed about the outer surface of said introducer needle and axially movable thereon between said retracted position and said deployed position.

9. The penetrating instrument of claim 1, wherein the outer surface of said introducer needle comprises an axial groove in which at least a portion of said piercing needle is disposed and axially movable therein between said retracted position and said deployed position.

10. The penetrating instrument of claim 1, further comprising:
an introducer member, wherein said introducer needle is in communication with and distally extending from the distal surface of said introducer member with said introducer member comprising a central region wherein said actuating member is disposed with said actuating member in communication with said piercing needle.

11. A penetrating instrument, comprising:
an introducer needle having a proximal end and a tapered distal end;
a piercing needle having a distal tip, said piercing needle in slidable communication with said introducer needle; and
an actuating member connected to said piercing needle, said actuating member axially advances said piercing needle a predetermined distance relative to said introducer needle wherein said piercing needle is actuated between a retracted position and a deployed position, said retracted position comprising said distal tip of said piercing needle disposed proximal to said distal end of said introducer needle and said deployed position comprising said distal tip of said piercing needle disposed said predetermined distance distal to said distal end of said introducer needle;
wherein said tapered distal end of said introducer needle allows for independent penetration of tissue by said introducer needle when said piercing needle is disposed in said retracted position;
wherein said actuating member is selected from the group consisting of a manual grip, a lever, a spring, and a motor activator and said actuating member further provides for retraction of said piercing needle from said deployed position to said retracted position.

12. The penetrating instrument of claim 11, wherein said introducer needle comprises a central hollow channel in which said piercing needle is disposed and axially movable therein between said retracted position and said deployed position.

13. The penetrating instrument of claim 11, wherein said piercing needle is disposed about the outer surface of said introducer needle and axially movable thereon between said retracted position and said deployed position.

14. The penetrating instrument of claim 11, wherein the outer surface of said introducer needle comprises an axial groove in which at least a portion of said piercing needle is disposed and axially movable therein between said retracted position and said deployed position.

15. A method for penetrating selected tissue, comprising the steps of:
providing a penetrating instrument comprising:
an introducer needle having a proximal end and a tapered distal end;
a piercing needle having a distal tip, said piercing needle in slidable communication with said introducer needle; and
an actuating member connected to said piercing needle, said actuating member axially advances said piercing needle a predetermined distance relative to said introducer needle wherein said piercing needle is actuated between a retracted position and a deployed position, said retracted position comprising said distal tip of said piercing needle disposed proximal to said distal end of said introducer needle, said deployed position comprising said distal tip of said piercing needle disposed said predetermined distance distal to said distal end of said introducer needle;
wherein said tapered distal end of said introducer needle allows for independent penetration of tissue by said introducer needle when said piercing needle is disposed in said retracted position;
wherein said actuating member is selected from the group consisting of a manual grip, a lever, a spring, and a motor activator and said actuating member further provides for retraction of said piercing needle from said deployed position to said retracted position;
applying an advancement force to said introducer needle;
advancing said introducer needle through tissue until said distal end of said introducer needle is disposed immediately adjacent to said selected tissue, wherein said piercing needle is disposed in said retracted position;
applying a first activation force to said piercing needle; and
advancing said piercing needle from said retracted position to said deployed position, wherein said piercing needle is advanced said predetermined distance into said selected tissue.

16. The method of claim 15, further comprising the steps of:
applying a retraction force to said piercing needle, wherein said piercing needle is moved from said deployed position to said retracted position;
applying a second activation force to said piercing needle; and
advancing said piercing needle a second time from said retracted position to said deployed position, wherein said piercing needle is advanced said predetermined distance into said selected tissue.

17. The method of claim 15, further comprising the steps of:
reapplying said advancement force to said introducer needle, wherein said piercing needle is maintained in said deployed position; and
advancing said introducer needle through said selected tissue.

18. The method of claim 15, further comprising the steps of:
applying a retraction force to said piercing needle, wherein said piercing needle is moved from said deployed position to said retracted position;
reapplying said advancement force to said introducer needle, wherein said piercing needle is maintained in said retracted position.
advancing said introducer needle through said selected tissue which has already been penetrated by piercing needle to predetermined distance.

19. The method of claim 15, wherein said introducer needle comprises a central hollow channel in which said piercing needle is disposed and axially movable therein between said retracted position and said deployed position.

20. The method of claim 15, wherein said piercing needle is disposed about the outer surface of said introducer needle and axially movable thereon between said retracted position and said deployed position.

21. The method of claim 15, wherein the outer surface of said introducer needle comprises an axial groove in which at least a portion of said piercing needle is disposed and axially movable therein between said retracted position and said deployed position.
